(19) United States
(12) Patent Application Publication
(10) Pub. No.: US 2005/0139490 A1
(43) Pub. Date: Jun. 30, 2005

(54) Alkaloid sensor, systems comprising the same, and measurement using the systems

(75) Inventors: Jung-Chuan Chou, Yunlin Hsian (TW); Shin-Cheng Chang, Nantou County (TW)

Correspondence Address: BIRCH STEWART KOLASCH & BIRCH, PO BOX 747, FALLS CHURCH, VA, 22040-0747, US

(73) Assignee: National Yunlin University of Science and Technology

(21) Appl. No.: 11/023,506

(22) Filed: Dec. 29, 2004

(30) Foreign Application Priority Data
Dec. 30, 2003 (TW) 92137406

(51) Int. Cl. G01N 33/00; G01F 1/64; G01N 17/04

(52) U.S. Cl. 205/777; 436/96; 436/106

(57) ABSTRACT

An alkaloid sensor, systems comprising the same, and measurement using the systems. The alkaloid sensor has an extended gate field effect transistor (EGFET) structure and comprises a metal oxide semiconductor field effect transistor (MOSFET) on a semiconductor substrate, a sensing unit comprising a substrate, a tin oxide film on the substrate, and a alkaloid acylase film immobilized on the tin oxide film, and a conductive wire connecting the MOSFET and the sensing unit.

### BACKGROUND

The invention relates to a sensor, and more specifically to a sensor measuring alkaloid concentration.

Berberine (BE) is an alkaloid derived from berberis aristata. Berberine is an antiseptic medicine which inhibits streptococcus, staphylococcus, and shigella. Berberine is also used as a stomach and bowel medicine. Recently, berberine is reported to promote anti-tumor activities and anti-lipase effect. The conventional methods for detecting berberine quantities comprise acetone-berberine precipitation and film fluorescence scanning.

A sensor for detecting berberine quantities is disclosed, in Li Xianwen and Huang Qiang, “Development and Application of Silicotungistic Acid-Berberine Field Effect Transistor”, Chinese Journal of Analytical Chemistry, 1997, vol. 25(11), pp.1297-1299. The sensor utilizes an ion sensitive field effect transistor (ISFET). An active material is immobilized on a sensing window of a sensing unit by polymer entrapment. The active material is silicotungistic acid, mixed with PVC and diocyl phthalate. The test results obtained agree with the pharmacopeia method.

Another method for detecting ephedrine quantities is disclosed, in Li Xianwen and Huang Qiang, “Development and Application of Silicotungistic Acid-Ephedrine Field Effect Transistor”, Chinese Journal of Analytical Chemistry, 1998, vol. 17(3), pp. 90-92. The sensor provided by the method has a higher sensitivity than that comprising an ion selective electrode.

The above-mentioned ion sensitive field effect transistors, however, have several drawbacks, such as interference with test solutions due to contact with each other.

### SUMMARY

The invention provides a sensor for measuring alkaloid concentration. The sensor comprises an extended gate ion sensitive field effect transistor and a tin oxide sensitive film having a silicotungstic acid film immobilized thereon to detect alkaloid concentration in a solution. The invention provides low cost, high sensitivity of ion sensitive films, accurate measurement, and rapid response time. A sensing unit is isolated from a field effect transistor to prevent unstable characteristics on semiconductor elements and decrease interference from test solutions. Additionally, polymer entrapment combined with an extended gate ion sensitive field effect transistor has advantages of simple package, easy storage, isolation from photosensitive effect, and unrestricted sensing window size. The polymer entrapment is also suitable for preparing a disposable sensor.

The invention provides a system comprising an alkaloid sensor and measurement using the system to measure response curves of reaction time and recovery time of the alkaloid sensor.

The invention provides an alkaloid sensor having an extended gate field effect transistor structure comprising a metal oxide semiconductor field effect transistor (MOSFET), a sensing unit comprising a substrate, a tin oxide film on the substrate, and a silicotungstic acid film immobilized on the tin oxide film, and a conductive wire connecting the MOSFET and the sensing unit.

The invention provides a system of measuring alkaloid concentration in a solution, comprising the above-mentioned alkaloid sensor, a reference electrode supplying stable voltage, a semiconductor characteristic instrument connecting the alkaloid sensor and the reference electrode, respectively, a temperature controller comprising a temperature control center, a thermocouple, a heater, and a light-isolation container isolating the sensing unit from photosensitive effect, wherein the temperature control center connects the thermocouple and the heater, respectively. Measurement of alkaloid concentration in a solution comprises pouring a solution into the light-isolation container, immersing the alkaloid sensor, the reference electrode, and the thermocouple in the solution, adjusting temperature of the solution by the heater controlled by the temperature control center after detecting temperature variation in the solution by the thermocouple, transmitting measurement data from the alkaloid sensor and the reference electrode to the semiconductor characteristic instrument, and reading out current-voltage (I-V) values of the solution by the semiconductor characteristic instrument to obtain alkaloid concentration in the solution.

The invention provides a method of measuring sensitivity of an alkaloid sensor, using the above-mentioned system, comprising immersing the silicotungstic acid film of the alkaloid sensor in an alkaloid solution, recording a curve of source/drain current versus gate voltage of the alkaloid sensor by the semiconductor characteristic instrument after altering pH values of the alkaloid solution at a fixed temperature, and examining the curve to obtain a sensitivity of the alkaloid sensor at the fixed temperature and a fixed current.

The invention also provides a system of measuring alkaloid concentration in a solution, comprising the above-mentioned alkaloid sensor, a reference electrode supplying a stable voltage, an instrumentation ampilfier having two inputs and one output, a high-resistance multimeter connecting the output of the instrumentation ampilfier, and a microcomputer pH meter, wherein the two inputs connects the alkaloid sensor and the reference electrode, respectively. The steps of measuring alkaloid concentration in a solution comprises determining a pH value of a solution by the microcomputer pH meter, immersing the alkaloid sensor and the reference electrode in the solution, and reading out a response voltage of the sensing unit by the high-resistance multimeter to obtain alkaloid concentration in the solution.

The invention further provides a method of measuring a response of a alkaloid sensor, using the above-mentioned system, comprising measuring a pH value of a alkaloid solution by the microcomputer pH meter, immersing the alkaloid acylase film of the alkaloid sensor in the alkaloid solution, recording an output voltage of the alkaloid sensor by the high-resistance multimeter, and altering concentration of the alkaloid solution and repeating the first four steps to obtain a response of the alkaloid sensor. The response is an output voltage variation between initial and terminal measuring points at a fixed pH value.

A detailed description is given in the following embodiments with reference to the accompanying drawings.

### BRIEF DESCRIPTION OF THE DRAWINGS

Embodiments of the invention can be more fully understood by reading the subsequent detailed description and examples with references made to the accompanying drawings, wherein:

FIG. 1 is a cross-section of an extended gate field effect transistor of the invention.

FIG. 2 is a cross-section of a conventional ion sensitive field effect transistor.

FIG. 3 is a cross-section of a drug sensor having a silicotungistic acid film immobilized thereon of the invention.

FIG. 4 shows a current-voltage system of measuring a sensitivity of a tin oxide film of the invention.

FIG. 5 shows a sensing unit and a readout circuit.

FIG. 6 shows the sensitivity of a tin oxide sensitive film in berberine solutions with various pH of the invention.

FIG. 7 shows voltage curves of an alkaloid sensor in berberine solutions of various concentrations of the invention.

FIG. 8 shows an optimal linear sensitivity of an alkaloid sensor in a berberine solution of the invention.

FIG. 9 shows response time of an alkaloid sensor of the invention.

### DETAILED DESCRIPTION

Referring to FIG. 2, a conventional ion sensitive field effect transistor (ISFET) comprises a p-type silicon substrate 13, a gate comprising a silicon dioxide film 11 on the substrate, and a sensitive film 10 immobilized on the film 11, wherein only the sensitive film 10 can directly contact a test solution 7. Other elements of the ISFET are covered by an insulation region 8 comprising epoxy resin. Both sides of the silicon dioxide film 11 in the substrate are n-type heavy doped regions (source/drain) 12. A conductive wire 9, such as aluminum wire, connects the transistor such that source/drain electronic signals can be transmitted to additional circuits thereby after the test solution 7 is detected by the sensitive film 10. Additionally, a reference electrode 14 calibrates measured data.

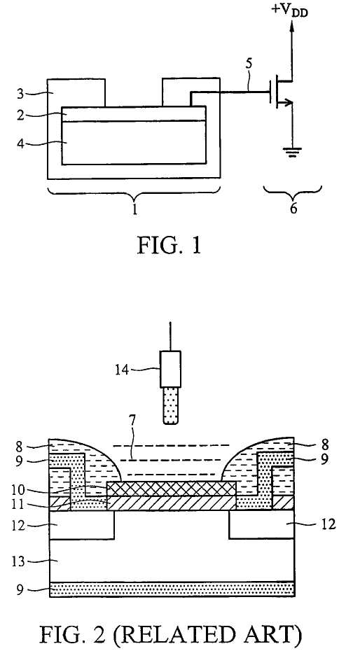

An extended gate field effect transistor (EGFET) is developed from an ISFET. A sensitive film is isolated from a gate of an ISFET, that is, a metal oxide semiconductor field effect transistor (MOSFET) is completely isolated from a test solution to prevent unstable characteristics on semiconductor elements and decrease interference from the test solution. Referring to FIG. 1, an extended gate field effect transistor comprises a sensing unit 1 and a MOSFET 6, wherein the sensing unit 1 comprises a conductive glass 4, such as indium tin oxide (ITO) glass, and a tin oxide film 2 on the conductive glass 4. A conductive wire 5 connects the sensing unit 1 and the gate of the MOSFET 6. The sensing unit 1 is covered by an insulation region 3, exposing partial tin oxide film 2 to contact a test solution (not shown). Detection by an EGFET is described as follows. First, adsorbent hydrogen ions of a tin oxide sensitive film are converted to electronic signals. Channel width of a MOSFET is then controlled by the electronic signals. Finally, hydrogen ion concentration is obtained by examining current values.

Berberine may react with silicotungistic acid solution to produce white precipitates. The invention provides an alkaloid sensor, wherein alkaloid is not limited to berberine, further comprising ephedrine. The invention provides polymer entrapment to immobilize an active material, such as silicotungistic acid, on a sensing window of an extended gate sensitive field effect transistor, since an alkaloid, a weak base containing nitrogen atoms, such as berberine, may react with an acidic material, such as silicotungistic acid, to form a salt. The plasticizer provided by the invention comprises diocyl phthalate. The polymer provided by the invention comprises polyvinyl chloride (PVC) or a polymer having the same solubility parameters as a plasticizer. In polymer entrapment, PVC, silicotungistic acid, and diocyl phthalate are mixed in a preferable ratio of 4:1.5:3, and added to a solvent, such as tetrahydrofuran, wherein the solvent is an evaporated solvent, with amount of the solvent corresponding to thickness of the sensitive film. The polymer entrapment is simple and low cost. When silicotungistic acid reacts with an alkaloid solution, such as a berberine solution, hydrogen ions of the active silicotungistic acid may be separated into the solution, leaving metal ions trapped by polymers. The metal ions then gradually combine with alkaloid molecules and form stable bonding therewith. The coupling molecules in the sensitive film have the same direction. The surface of the sensitive film may represent a stable bonding potential so that electrochemical energy can be converted to an electrical signal by the field effect transistor. According to the characteristics described above, the invention provides a sensor combining an active material and an extended gate sensitive field effect transistor.

The current-voltage system shown in FIG. 4 measures sensitivity of an alkaloid sensor. A sensing unit 20 of an alkaloid sensor is immersed in a test solution 24 placed in a container (not shown). A semiconductor characteristic instrument 21, such as HP4155B, connects a source and a drain of the sensing unit 20 by conductive wires 28 and 29, such as aluminum wire, to process electronic signals.

Additionally, a reference electrode 22 is immersed in the test solution 24 to supply a stable voltage. The reference electrode 22 is an Ag/AgCl reference electrode. The reference electrode 22 connects the semiconductor characteristic instrument 21 by a conductive wire 30. A set of heaters 25 is installed outside the container, connecting a temperature controller 26 (temperature control center). When temperatures of the test solution 24 are altered, the temperature controller 26 may drive the heaters 25 to adjust the test solution temperature, wherein a thermocouple 27 of the temperature controller 26 detects the temperature of the test solution 24. The test solution 24, the heaters 25, and other elements contacting the test solution 24 are placed in a light-isolation container 23, such as a dark box, to prevent photosensitive effect.

The method of measuring a sensitivity of an alkaloid sensor using the above-mentioned system is described in the following. First, the silicotungistic acid film of the alkaloid sensor is immersed in an alkaloid solution, such as a berberine solution. Subsequently, pH values of the alkaloid solution are altered from 2 to 10 at a fixed temperature, generally 25° C. Next, the semiconductor characteristic instrument supplies a voltage from 1 to 6V to the gate of the alkaloid sensor, and supplies a fixed voltage of 0.2V to the source/drain thereof. Next, a curve of source/drain current versus gate voltage of the alkaloid sensor is recorded by the semiconductor characteristic instrument 21. Finally, the curve is examined to obtain a sensitivity of the alkaloid sensor at the fixed temperature and a fixed current.

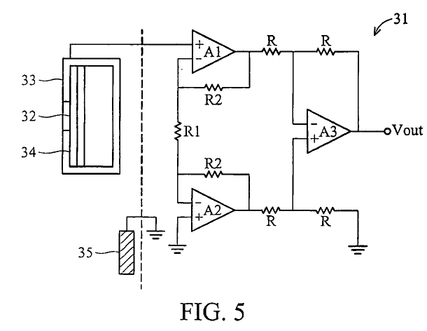

Additionally, electronic signals of the alkaloid sensor 33 (comprising silicotungistic acid film 32 and a tin oxide film 34) may be amplified, as shown in FIG. 5, and measured data of various test solutions can be read by an instrumentation ampilfier 31. A reference electrode 35 calibrates the measured data. The system comprises the above-mentioned alkaloid sensor, a reference electrode, such as an Ag/AgCl reference electrode, supplying a stable voltage, an instrumentation ampilfier, such as LT1167, having two inputs and one output, a high-resistance multimeter, such as HP3478A, and a microcomputer pH meter having a pH measuring range from 1 to 14 and a resolution of 0.01, wherein the alkaloid sensor and the reference electrode connect the two inputs, respectively, and the high-resistance multimeter connects the output of the instrumentation ampilfier. Measurement of alkaloid concentration in a solution using the above-mentioned system are described in the following. First, a pH value of a solution is determined by the microcomputer pH meter. Next, the alkaloid sensor and the reference electrode are immersed in the solution. Finally, current-voltage values of the solution are read out by the high-resistance multimeter to obtain alkaloid concentration in the solution.

### EXAMPLE

Manufacture of the Alkaloid Sensor

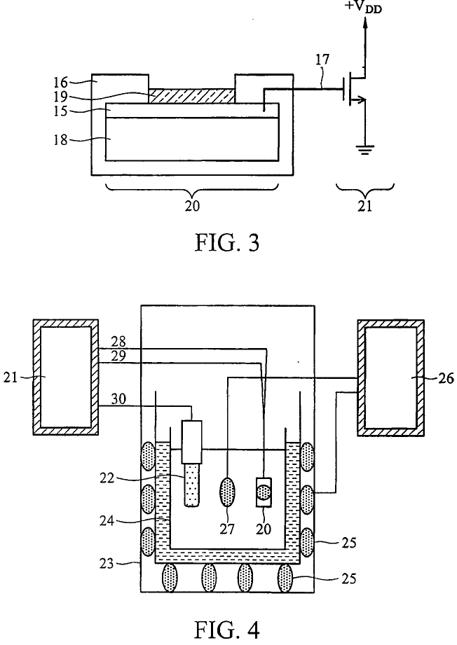

Referring to FIG. 3, a cross-section of an alkaloid sensor is illustrated. First, a 1.5 cm×1.5 cm tin oxide film 15 was prepared on an ITO glass 18 by RF sputtering to form a sensing unit. The sensing unit was covered by epoxy resin 16, exposing partial tin oxide film 19 to form a sensing window of about 2 mm×2 mm. The sensing unit was connected with a gate of a MOSFET by an aluminum wire 17.

After the sensing unit and the transistor were packaged, active silicotungistic acid 19 was immobilized on the tin oxide film 15 by PVC entrapment. The steps of immobilization are described in the following. First, 1.5 g silicotungistic acid, 4 g PVC, and 3 ml diocyl phthalate were mixed and added to a THF solution. Next, the solution was stirred to dissolve completely. 1 μl silicotungistic acid mixing solution was then dropped on the sensing window. After a drying period, the sensor was placed in a dry room at room temperature for 24 hrs. Finally, the drug sensor was immersed in a 1 mM berberine solution for activation for 24 hrs. The sensor was rinsed with 1 mM berberine solution for 10 min between each measurement.

Measurement of the Berberine Solution using the Current-Voltage Measuring System

The current-voltage measuring system of the invention is illustrated in FIG. 4. A sensing unit 20 and an Ag/AgCl reference electrode 22 were immersed in a test solution 24. A current-voltage curve of a sensor in the test solution was measured by a semiconductor characteristic instrument 21 (HP4155B). The temperature of the test solution was controlled at 25° C.

The readout circuit of the alkaloid sensor of the invention is illustrated in FIG. 5. An alkaloid sensor 33 and an Ag/AgCl reference electrode 35 were immersed in a test solution. Sensor response was obtained using a readout circuit.

Measurement of concentration of an alkaloid solution is described in the following. First, a test solution was cooled to room temperature. Next, an alkaloid sensor was immersed in a 1 mM berberine solution for 60 sec to determine the standard voltage value. Voltage values of various berberine test solutions were then measured. A voltage-time curve was then plotted by Microsoft Origin 6.0 according to the measuring data.

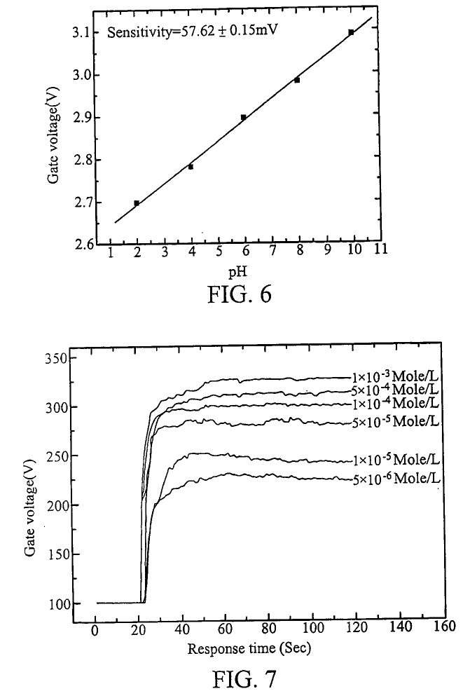

Finally, the sensitivity of the drug sensor of the invention was obtained by analyzing the curve. The sensitivity was about 57.62 mV/pH, as shown in FIG. 6. The linear detecting scope for a berberine solution of the alkaloid sensor was 1×10⁻³˜5×10⁻⁶ mM, as shown in FIG. 7. In FIG. 7, output voltages show high repeatability and stability even when the sensor was continually used for above 100 min.

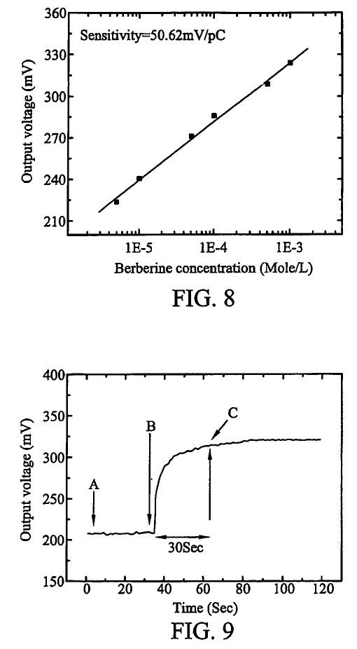

Before measurement, an alkaloid sensor is activated with concentrated berberine solution for about 24 hrs to maintain a stable response voltage on the surface thereof. Although the response voltages of various sensors show slight errors, the sensitivity thereof is the same. The output voltages may linearly decrease as concentration of test solutions decreases, as shown in FIG. 8.

Response time of a sensor can be measured using the system provided by the invention, as shown in FIG. 9. After an alkaloid sensor was rinsed with DI water, a berberine test solution was measured therewith. A response time of about 30 sec is obtained after the output voltage stabilizes.

The alkaloid sensor of the invention has advantages of high sensitivity, accurate measurement, rapid response time, and high repeatability and stability for long-term utilization. Additionally, the invention may also provide a disposable drug sensor.

While the invention has been described by way of example and in terms of the preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. An alkaloid sensor with an extended gate field effect transistor structure, comprising:
a metal oxide semiconductor field effect transistor (MOSFET) on a semiconductor substrate;
a sensing unit comprising a substrate, a tin oxide film on the substrate, and a silicotungstic acid film immobilized on the tin oxide film; and
a conductive wire connecting the MOSFET and the sensing unit.

2. The alkaloid sensor as claimed in claim 1, wherein the metal oxide semiconductor field effect transistor is an N-type field effect transistor.

3. The alkaloid sensor as claimed in claim 1, wherein the conductive wire connects a gate of the metal oxide semiconductor field effect transistor and the sensing unit.

4. The alkaloid sensor as claimed in claim 1, wherein the substrate is a conductive glass.

5. The alkaloid sensor as claimed in claim 4, wherein the substrate is an indium tin oxide glass.

6. The alkaloid sensor as claimed in claim 1, wherein the silicotungstic acid film is immobilized on the tin oxide film by polymer entrapment.

7. The alkaloid sensor as claimed in claim 6, wherein the silicotungstic acid film is formed by mixing polymers, plasticizers, and silicotungstic acid in a solvent.

8. The alkaloid sensor as claimed in claim 7, wherein the polymer comprises polyvinyl chloride (PVC).

9. The alkaloid sensor as claimed in claim 7, wherein the plasticizer comprises diocyl phthalate.

10. The alkaloid sensor as claimed in claim 7, wherein the solvent comprises tetrahydrofuran (THF).

11. The alkaloid sensor as claimed in claim 7, wherein the polymer, the silicotungstic acid, and the plasticizer are in a ratio of 4:1.5:3.

12. The alkaloid sensor as claimed in claim 1, further comprising an epoxy resin on the surface of the sensing unit.

13. A system of measuring a concentration of alkaloid in a solution, comprising:
an alkaloid sensor as claimed in claim 1;
a reference electrode supplying a stable voltage;
a semiconductor characteristic instrument connecting the alkaloid sensor and the reference electrode, respectively;
a temperature controller comprising a temperature control center, a thermocouple, and a heater, wherein the temperature control center connects the thermocouple and the heater, respectively; and
a light-isolation container isolating the sensing unit from photosensitive effect, wherein Measurement of a concentration of alkaloid in a solution comprises pouring a solution into the light-isolation container, immersing the alkaloid sensor, the reference electrode, and the thermocouple in the solution, adjusting temperatures of the solution by the heater controlled by the temperature control center after detecting temperature variations of the solution by the thermocouple, transmitting measured data of the alkaloid sensor and the reference electrode to the semiconductor characteristic instrument, and reading out current-voltage (I-V) values of the sensing unit by the semiconductor characteristic instrument to obtain a concentration of alkaloid in the solution.

14. The system as claimed in claim 13, wherein the alkaloid comprises berberine.

15. The system as claimed in claim 13, wherein the alkaloid comprises ephedrine.

16. The system as claimed in claim 13, wherein the semiconductor characteristic instrument is HP4155B.

17. The system as claimed in claim 13, wherein the solution has a temperature of 25° C. controlled by the temperature controller.

18. The system as claimed in claim 13, wherein the reference electrode is an Ag/AgCl reference electrode.

19. The system as claimed in claim 13, wherein the light-isolation container is a dark box.

20. A method of measuring a sensitivity of an alkaloid sensor, using the system as claimed in claim 13, comprising:
(a) immersing a silicotungstic acid film of an alkaloid sensor as claimed in claim 1 in an alkaloid solution;
(b) recording a curve of source/drain current versus gate voltage of the alkaloid sensor by the semiconductor characteristic instrument after altering pH values of the alkaloid solution at a fixed temperature; and
(c) examining the curve to obtain a sensitivity of the alkaloid sensor at the fixed temperature and a fixed current.

21. The method as claimed in claim 20, wherein the alkaloid comprises berberine.

22. The method as claimed in claim 20, wherein the alkaloid comprises ephedrine.

23. The method as claimed in claim 20, wherein the alkaloid solution has a pH value from 2 to 10.

24. The method as claimed in claim 20, wherein the semiconductor characteristic instrument supplies a voltage from 1 to 6V to a gate of the metal oxide semiconductor field effect transistor of the alkaloid sensor.

25. The method as claimed in claim 22, wherein the semiconductor characteristic instrument supplies a fixed voltage of 0.2V to a source/drain of the metal oxide semiconductor field effect transistor of the alkaloid sensor.

26. The method as claimed in claim 22, wherein the alkaloid solution has a temperature of 25° C. controlled by the temperature controller.

27. The method as claimed in claim 22, wherein the reference electrode is an Ag/AgCl reference electrode.

28. A system of measuring a concentration of alkaloid in a solution, comprising:
an alkaloid sensor as claimed in claim 1;
a reference electrode supplying a stable voltage;
an instrumentation ampilfier having two inputs and one output, wherein the two inputs connect the alkaloid sensor and the reference electrode, respectively;
a high-resistance multimeter connecting the output of the instrumentation ampilfier; and
a microcomputer pH meter, wherein Measurement of a concentration of alkaloid in a solution comprises determining a pH value of a solution by the microcomputer pH meter, immersing the alkaloid sensor and the reference electrode in the solution, and reading out current-voltage values of the solution by the high-resistance multimeter to obtain a concentration of alkaloid in the solution.

29. The system as claimed in claim 28, wherein the alkaloid comprises berberine.

30. The system as claimed in claim 28, wherein the alkaloid comprises ephedrine.

31. The system as claimed in claim 28, wherein the high-resistance multimeter is HP3478A.

32. The system as claimed in claim 28, wherein the instrumentation ampilfier is LT1167.

33. The system as claimed in claim 28, wherein the microcomputer pH meter has a pH range from 1 to 14, and a resolution of 0.01.

34. The system as claimed in claim 28, wherein the reference electrode is an Ag/AgCl reference elctrode.

35. A method of measuring a response of an alkaloid sensor, using the system as claimed in claim 28, comprising:
(a) measuring a pH value of an alkaloid solution by the microcomputer pH meter;
(b) immersing the silicotungstic acid film of the alkaloid sensor as claimed in claim 1 in the alkaloid solution;
(c) recording a output voltage of the alkaloid sensor by the high-resistance multimeter; and
(d) altering concentrations of the alkaloid solution and repeating the steps (a)-(c) to obtain a response of the alkaloid sensor.

36. The method as claimed in claim 35, wherein the alkaloid comprises berberine.

37. The method as claimed in claim 35, wherein the alkaloid comprises ephedrine.

38. The method as claimed in claim 35, wherein the response is an output voltage variation between an initial and a terminal measuring points at a fixed pH.

39. The method as claimed in claim 35, wherein the reference electrode is an Ag/AgCl reference elctrode.
